(19) United States
(12) Patent Application Publication
(10) Pub. No.: US 2023/0398308 A1
(43) Pub. Date: Dec. 14, 2023

(54) MEDICAMENT DELIVERY DEVICE WITH PIEZOELECTRIC ACTUATING SYSTEM

(71) Applicant: SHL MEDICAL AG, ZUG (CH)

(72) Inventor: Nurettin Ali, Boca Raton, FL (US)

(21) Appl. No.: 18/035,995

(22) PCT Filed: Oct. 29, 2021
(86) PCT No.: PCT/EP2021/080153

§ 371 Date: May 9, 2023

Related U.S. Application Data
(60) Provisional application No. 63/114,147, filed on Nov. 16, 2020.

(30) Foreign Application Priority Data
Dec. 21, 2020 (EP) 20215880.4

(51) Int. Cl. A61M 5/315

(52) U.S. Cl. CPC A61M 5/31578

(57) ABSTRACT

An actuating system for a medicament delivery device is disclosed. The actuating system includes a first clamp arranged around a plunger of the medicament delivery device for selectively retaining and releasing the plunger. A piezoelectric actuator is coupled to the first clamp for moving the plunger via a plurality of consecutive steps. An electric controller is configured for controlling the piezoelectric actuator and the first clamp independently to coordinate the moving of the plunger.

### CROSS REFERENCE TO RELATED APPLICATIONS

The present application is a U.S. National Phase Application pursuant to 35 U.S.C. § 371 of International Application No. PCT/EP2021/080153 filed Oct. 29, 2021, which claims priority to U.S. Provisional Patent Application No. 63/114,147 filed Nov. 16, 2020 and European Patent Application No. 20215880.4, filed Dec. 21, 2020. The entire disclosure contents of these applications are herewith incorporated by reference into the present application.

### TECHNICAL FIELD

This present disclosure relates to a piezoelectric actuating system for a medicament delivery device such as an automatic injection device.

### BACKGROUND

Self-injection continues to be one of the most important methods of medicament delivery. An increasing number of high-viscosity therapeutic compositions are available for treating medical conditions such as auto-immune disease, cardiovascular disease, blood disorder and the like. Current automatic injectors fall short of meeting the need. For example, most state-of-the-art automatic injectors use coil-spring structures to provide the actuation energy needed for needle insertion and medicament delivery. A spring-loaded drive mechanism can be suitable for this purpose, provided that the viscosity of medicament is low. The force generated by a spring load is often predetermined and cannot be adjusted by the user. Therefore, it is difficult to use the traditional coil-spring structure to expel high-viscosity medicament or achieve customized medicament delivery. There is a desire to develop alternative actuation systems for generating adequate and adjustable output forces to deliver viscous or concentrated therapeutic formulations.

### SUMMARY

An actuating system for a medicament delivery device and a medicament delivery device employing the actuating system are provided. In an example embodiment, the actuating system includes (i) a first clamp arranged around a plunger of the medicament delivery device for selectively retaining and releasing the plunger; (ii) a piezoelectric actuator coupled to the first clamp for moving the plunger via a plurality of consecutive steps; and (iii) an electric controller configured for controlling the piezoelectric actuator and the first clamp independently to coordinate the moving of the plunger.

In another example embodiment, a medicament delivery device includes a syringe barrel having a reservoir therein for holding a medicament. A nozzle is disposed at a first end of the syringe barrel with at least one opening for allowing medicament to be expelled from the reservoir. A plunger is received at a second end of the syringe barrel. An actuating system is configured for ejecting medicament from the reservoir. The actuating system includes (i) a piezoelectric actuator operatively connected to the plunger for moving of the plunger; (ii) a first clamp arranged around the plunger for selectively retaining and releasing the plunger; and (iii) an electric controller configured for controlling the piezoelectric actuator and the first clamp independently to coordinate the moving of the plunger.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE DRAWINGS

Exemplary embodiments are described herein with reference to the drawings, in which:

FIG. 1A is a perspective view of an example piezoelectric actuating system, according to an example embodiment of the present disclosure.

FIG. 1B is a perspective view of an exemplary medicament delivery device.

FIG. 1C is an exploded view of the medicament device of FIG. 1B.

FIG. 2A is a cross-sectional view of the example piezoelectric actuating system of FIG. 1A at plunger advancement stage, according to an example embodiment of the present disclosure.

FIG. 2B is a cross-sectional view of the example piezoelectric actuating system of FIG. 1A at the first clamp release stage, according to an example embodiment of the present disclosure.

FIG. 2C is a cross-sectional view of the example piezoelectric actuating system of FIG. 1A at the first clamp retraction stage, according to an example embodiment of the present disclosure.

FIG. 3 is a perspective view of an example piezoelectric actuating system, according to another example embodiment of the present disclosure.

FIG. 4 is a perspective view of an example piezoelectric actuating system, according to yet another example embodiment of the present disclosure.

FIG. 5 is a perspective view of an example piezoelectric actuating system, according to yet another example embodiment of the present disclosure.

FIG. 6 is a perspective view of an example medicament delivery device without housing parts, employing the actuating system of FIG. 1A.

FIG. 7 is another perspective view of an example medicament delivery device without housing parts employing the actuating system of FIG. 5.

FIG. 8 is a perspective view of an example medicament delivery device without housing parts employing the actuating system of FIG. 4.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The methods and systems in accordance with the present disclosure beneficially provide improved methods and systems for propelling a plunger forward so as to eject the medicament from an injection device. The disclosed methods and systems provide a reliable and effective drug delivery device that uses a piezoelectric actuating system to eject a dose of medicament. Further, the disclosed methods and systems provide a versatile means for propelling the plunger forward so as to eject the medicament with a desired and reliable force and/or speed.

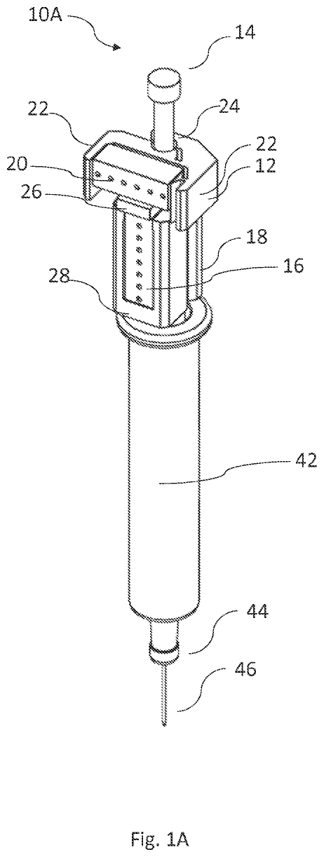

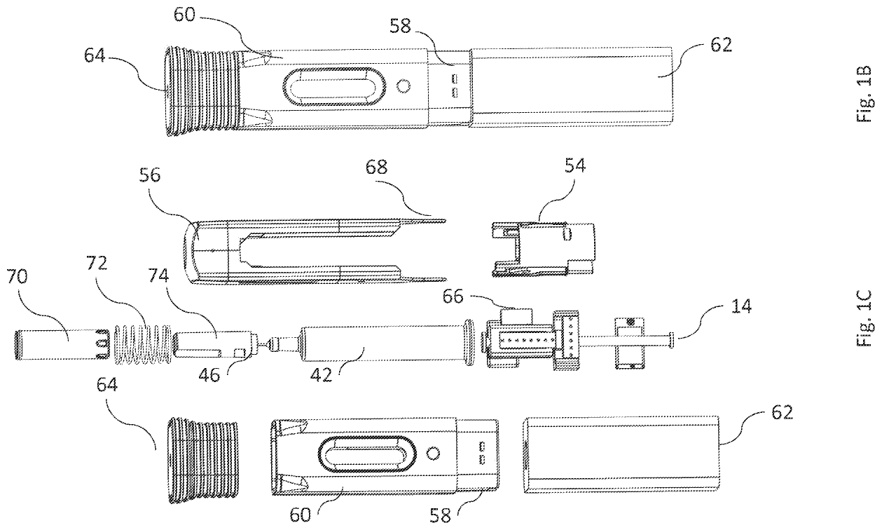

With reference to FIG. 1, in accordance with an example embodiment of the present disclosure, a piezoelectric actuating system 10A comprises a first clamp 12 arranged around a plunger 14 of a medicament delivery device for selectively retaining and releasing the plunger 14. The piezoelectric actuating system further comprises a first piezo stack actuator 20 and a second piezo stack actuator 16. The second piezo stack actuator 16 is connected to the first clamp 12 and configured for moving the plunger 14 via a plurality of consecutive steps. The system also comprises an electric controller 18 which is configured for controlling the first and the second piezo stack actuators. The electric controller controls the second piezo stack actuator 16 independently of the first clamp 12 to coordinate movement of the plunger 14.

In the depicted embodiment, the first clamp 12 is operated by the first piezo stack actuator 20. Specifically, the first piezo stack actuator 20 is securely fitted inside two arms 22 of the first clamp 12, and the jaw 24 of the first clamp 12 is configured to surround the plunger 14. When the first piezo stack actuator 20 is expanded via the electric controller 18, the two arms 22 are pushed outward, enabling the jaw 24 to be closed, thereby retaining the plunger 14. When the first piezo stack actuator 20 is contracted via the electric controller 18, the two arms 22 are pulled inward, enabling the jaw 24 to release the plunger 14. The first clamp 12 is coupled to the second piezo stack actuator 16. In the depicted embodiment, the first clamp 12 is coupled (e.g., glued, mechanically connected) to the piezo stack actuator 16 via a base 26. The base 26 is also used for supporting the first stack actuator 20 within the two arms 22.

In the depicted embodiment, the second stack actuator 16 is configured to expand and contract periodically via the electric controller 18. When the first stack actuator 20 is expanded, the arms 22 of the first clamp 12 are pushed outward, enabling the plunger 14 to be retained by the jaw 24. As such, contraction of the second stack actuator 16 can pull down the plunger 14 along with the first clamp 12. When the first stack actuator 20 is contracted, the arms 22 of the first clamp 12 are pulled inward, enabling the plunger 14 to be released by the jaw 24. As such, expansion of the second stack actuator 16 can push up the first clamp 12 without the plunger 14.

The actuating system 10A can further include a plunger holder 28. Placed around the plunger 14, the plunger holder 28 can be configured to provide resistance to the moving of the plunger 14 in a second direction (e.g., upward) while the plunger 14 is released by the first clamp 12 and when the first clamp 12 is moving in the second direction (e.g., upward). The resistance is used, for example, to counteract a reaction force generated by compressing the medicament, thereby retaining the plunger rod 14 in place when the first clamp 12 is being retracted and preparing for a next move. For example, as depicted in FIG. 1, the plunger holder 28 is designed to be placed around the plunger 14 and to hold the second piezo stack actuator 16. The friction between the plunger 14 and the plunger holder 28 can prevent movement of the plunger 14 along the second direction (e.g., upward) while the first clamp 12 is moving in the second direction (e.g., upward). This ensures a moving step of the plunger 14 in a first direction (e.g., downward) will not be compromised by movement of the first clamp 12 along the second direction (e.g., upward).

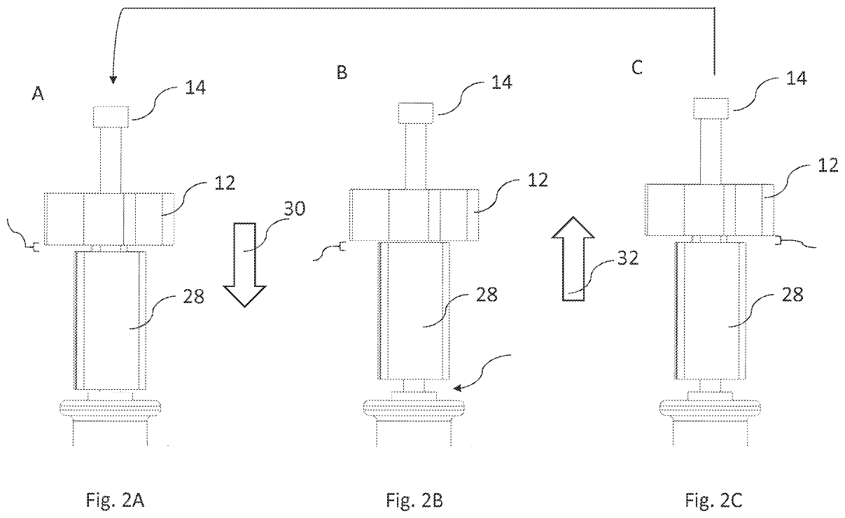

The actuation process is described in detail with reference to FIGS. 2A-2C. Referring to FIG. 2A, the first clamp 12 is configured to retain the plunger 14 via expanding the first piezo stack actuator 20, and the second piezo stack actuator 16 is contracted to pull down the plunger 14 together with the first clamp 12 for a predetermined distance (i.e., contraction distance of the second stack actuator 16) along the first direction 30 (medicament propulsion direction). The first clamp 12 is configured to release the plunger 14 via contracting the first piezo stack actuator 20, as shown in FIG. 2B, the second piezo stack actuator 16 is expanded to retract the first clamp 12 without the plunger 14 along the second direction 32 opposite to the first direction, as shown in FIG. 2C. The movement of the first clamp 12 in the second direction without the plunger 14 is ensured by the resistance between the plunger holder 28 and the plunger 14. By coordination of periodic expansion and contraction of the first and second piezo stack actuator 20 and 16, the process depicted in FIGS. 2A-2C is repeated in cycle. As such, the plunger 14 can be advanced to expel the medicament via a plurality of consecutive steps, each step having a predetermined distance. The distance depends on, at least partially, the voltage or current applied to the piezoelectric actuator 16 (e.g., second stack actuator 16), which will be discussed in detail with regard to the electric controller 18.

In another example embodiment, the actuating system can also be configured to retract the plunger 14, for example, after expelling a dose of medicament. Specifically, when the first clamp 12 is configured to retain the plunger 14 by expanding the first piezo stack actuator 20, and the second piezo stack actuator 16 is configured to be expanded, thereby pushing up the plunger 14 together with the first clamp 12 for a predetermined distance (i.e., expansion distance of the second stack actuator 16). The first clamp 12 is configured to release the plunger 14 by contracting the first piezo stack actuator 20, and the second piezo stack actuator 16 is configured to be contracted, thereby pulling down the first clamp 12 without the plunger 14. In this scenario, the plunger holder 28 is configured to resist the plunger 14 to move downward when the first clamp 12 is moving downward. As such, the coordination of the periodic expansion and contraction of the first and second piezo stack actuator 20 and 16 can retract the plunger 14 to its original position via a plurality of consecutive steps.

The electric controller 18 can be operable via an electrical power source (e.g., battery 34 or an AC power source). The electrical power source can be configured to generate a succession of electrical pulses to exert a high degree of accurate control over expelling a dose of medicament at a desired dosing rate. Parameters for the electric pulses can include a specific number, amplitude and width. The number of pulses can correspond to the number of steps of plunger movement. The amplitude of pulses can correspond to a moving distance of each moving step. The width of pulses can correspond to a frequency cycle of contraction and expansion of the first clamp 12 and the piezoelectric actuator 16 (e.g., second piezo stack actuator 16). All these factors can be used to control the position of the plunger 14 with high accuracy. As such, the electric controller 18 can be programmed to move the plunger 14 at a desired speed. For example, the desired speed can be achieved by adjusting the amplitude of electric pulses applied to the second piezo stack actuator 16, as this amplitude is associated with an extent of deformation of the second piezo stack actuator 16 and therefore a predetermined distance of each travel step of the plunger. As another example, the desired speed can be achieved by adjusting the frequency of expansion and contraction of the first and second piezo stack actuator 20 and 16, thereby adjusting the number of steps needed to expel a dose of medicament.

The electric controller 18 can be programmed via a user interface. The user interface can include one or more buttons or other types of touch-sensitive interface or display for allowing a user to set a dosage and/or a desired injection time. The user input can be converted to a corresponding amplitude of pulses, total number of pulses, and/or width of pulses.

It should be noted that as the movement of the plunger 14 needs to overcome the inertia of the liquid medicament during delivery, the time between ‘release’ and ‘retain’ of the plunger 14 will be greater than the time taken to move the plunger 14 in the first direction (e.g., direction to expel medicament from a syringe). This can vary based on the features of the medicament (e.g., viscosity of the medicament), syringe (e.g., syringe dimension) and hypodermic needle (e.g., diameter of the needle). As such, periodic change between ‘release’ signal and ‘hold’ signal controlled by the electric control 18 will be tailored according to specific medicament, syringe and needle.

It should also be noted that the first clamp 12 does not have to be operated via the first piezo stack actuator 20 as depicted in FIGS. 1 and 2A-2C. The first clamp 12 can be operated via pneumatic power, hydraulic power, magnetic force, or any other suitable clamping mechanism. Alternatively, the first clamp 12 itself can be made of piezoelectric material rather than operated by the first piezo stack actuator 20 placed between its two arms 22.

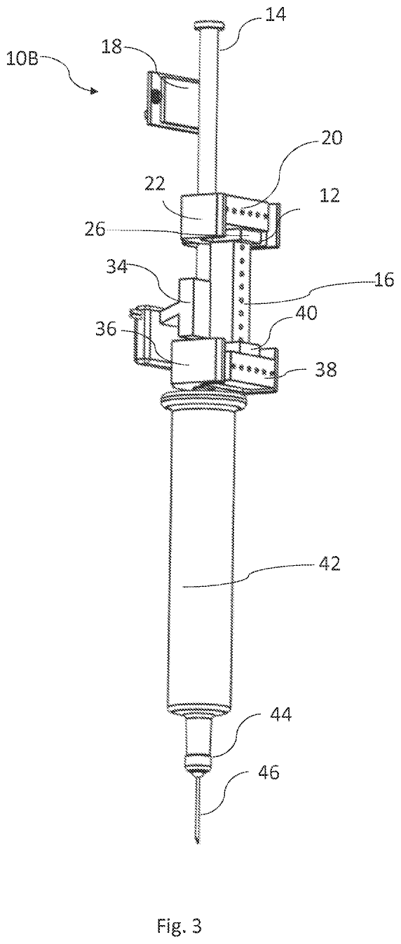

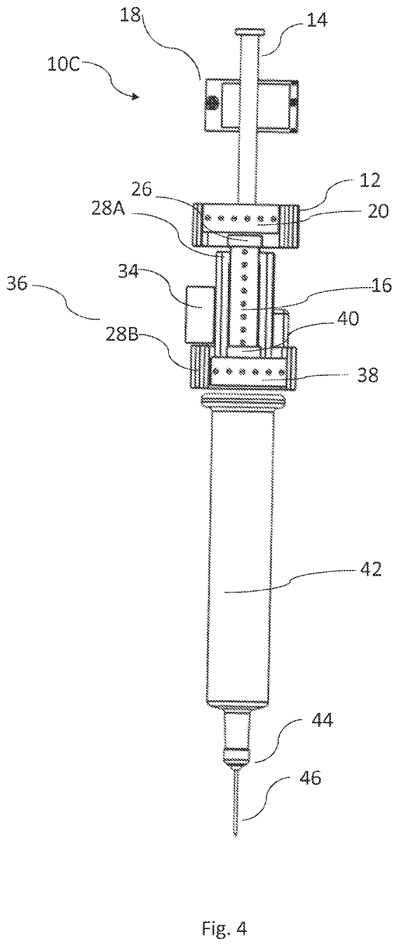

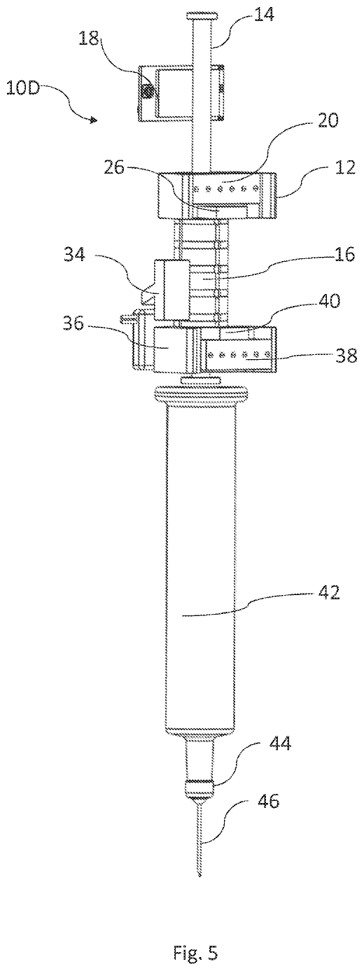

It should also be noted that the plunger holder 28 can differ from the embodiment depicted in FIG. 1. For example, as in the actuating system 10B shown in FIG. 3, the plunger holder can be a second clamp (e.g., second clamp 36) operated via a third piezo stack actuator 38. The second clamp 36 also includes a base 40 for coupling with the second piezo stack actuator 16. When the first clamp 12 is configured to release the plunger 14, the second clamp 36 is configured to retain the plunger 14 by expanding the third piezo stack actuator 38. As such, the first clamp 12 can be freely retracted (e.g., moving upward) while the plunger 14 is secured in place by the second clamp 36. Alternatively, as an actuating system 10C is shown in FIG. 4, the plunger holder includes two portions. A first portion, referred as 28A, is the plunger holder 28 depicted in FIG. 1. A second portion, referred as 28B, is the second clamp 36 depicted in FIG. 3. This type of design can provide greater retaining ability for the plunger 14 than the actuating system depicted in FIGS. 1 and 3.

It should also be noted that the shape of the piezo stack actuators (e.g., second stack actuator 16) does not have to be shaped as illustrated in FIGS. 1-4. For example, the second piezo stack actuator 16 can be a cylindrical-shaped piezo electric actuator configured to surround the plunger 14 for even distribution of the output force and thus provide more driving force than a rectangular shaped piezo stack actuator, as an actuating system 10D shown in FIG. 5.

With reference to FIGS. 1 and 3-5, the actuating system is shown to be incorporated with other elements of a medicament delivery device. Specifically, other elements of the medicament delivery device can include a syringe barrel 42 having a reservoir therein for holding a medicament. A nozzle 44 is disposed at a first end of the syringe barrel 42 with at least one opening for allowing medicament to be expelled from the reservoir. A needle 46 is connected to the nozzle 44. The plunger 14 is received at a second end of the syringe barrel 42. An actuating system (e.g., actuating system 10A, 10B, 10C and 10D) is employed in the medicament delivery device for ejecting the medicament from the reservoir.

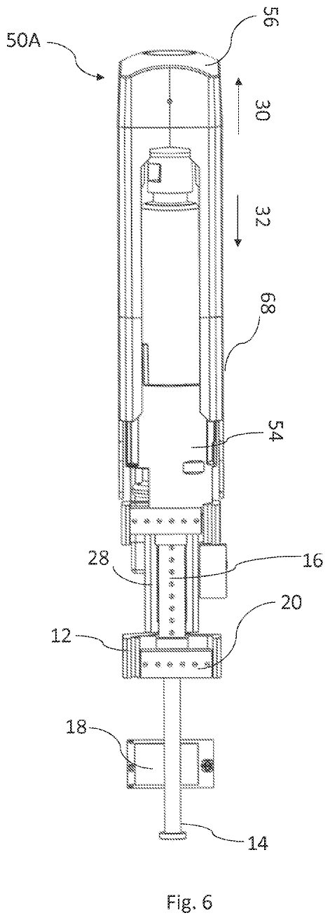

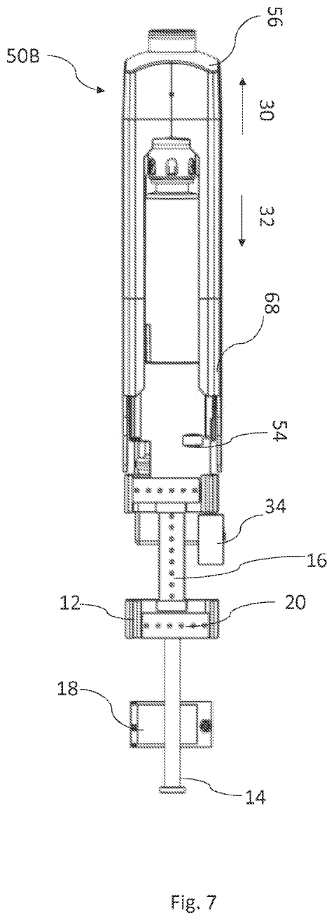

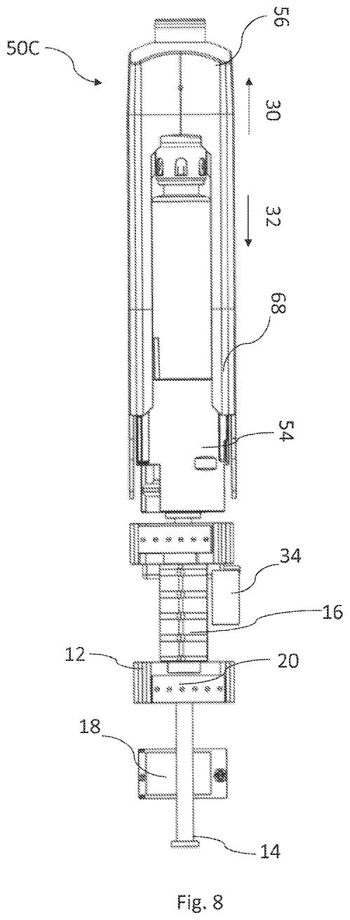

With reference to FIGS. 6-8 and 1B and 1C, more components are shown in respective medicament delivery devices. Specifically, a medicament delivery device (e.g. 50A, 50B, 50D) includes an actuating system (e.g., actuating system 10A, 10B, 10D), a syringe barrel (e.g., the syringe barrel 42) as shown in FIGS. 1 and 3-5, a syringe carrier (e.g., the syringe carrier 54), and a needle cover (e.g., needle cover 56) arranged in a main housing 58 as shown in FIGS. 1B and 1C. Main housing 58 includes a first housing portion 60 and a second housing portion 62. Although main housing 58 is depicted as comprising first and second housing portions 60 and 62, in other examples, main housing 58 may comprise more or fewer portions or can be of unitary construction. The medicament delivery device can also include an end cap (e.g., end cap 64) at the first end of the syringe barrel 42.

In an example embodiment, the movement of a needle cover 56 can activate the electric controller 18 via a switch 66. Specifically, to initiate the injection process, a user places the medicament delivery device (e.g., medicament delivery device 50A, 50B, 50D) on an injection site. When the medicament delivery device (e.g., 50A, 50B, 50D) is pressed onto the injection site in the first direction 30, the needle cover 56 moves in a second direction 32 relative to the main housing 58. The retraction of the needle cover 56 exposes a needle (not shown) and the needle is consequently inserted into the injection site. In addition to exposing needle, this retraction of the needle cover 56 can also serve to trigger the electric controller 18. In particular, the axial movement of the needle cover 56 in the second direction 32 causes axial movement of two arms 68 of the needle cover 56 in the second direction 32. During axial movement, one of the two arms 68 will trigger the switch 66 and activate the electric controller 18.

After injection is complete, the device (e.g., 50A, 50B, 50C) is removed from the injection site and the needle cover 56 will extend outward and lock into place. This extension and locking may limit or prevent needle stick injuries. The needle cover 56 may extend outward and lock into place in any suitable manner. For example, when the drug delivery device 50 is removed from the injection site, the needle cover 56 automatically extends outward in the first direction 30 under a force such as a spring force (not shown).

It should be noted that even though a syringe 42 is described in the figures, any suitable type of medicament container may be used, such as a syringe, an ampoule, a cartridge, an enclosure, and the like. Further, the medicament may be any suitable substance used for medical treatment.

The disclosed piezoelectric actuating system provides effective and versatile means for propelling a plunger forward in an automatic injection device via a consecutive of moving steps. Each moving step can be from 10 m to 30 m, which enables a high precision control of drug delivery. In addition, a high frequency cycle between the contraction and expansion state of the piezoelectric actuator can be achieved. This enables the plunger to travel 45 mm in under 10 s, and the output force can be up to 400 N. The disclosed piezoelectric actuating system is suitable for generating sufficient and adjustable output forces to deliver high viscosity or high concentration therapeutic formulations.

In the Figures, various engagement features for are shown for providing an engagement between one or more components of the drug delivery device. The engagement features may be any suitable connecting mechanism such as a snap lock, a snap fit, form fit, a bayonet, lure lock, threads or combination of these designs. Other designs are possible as well.

It should be understood that the illustrated components are intended as an example only. In other example embodiments, fewer components, additional components, and/or alternative components are possible as well. Further, it should be understood that the above described and shown embodiments of the present disclosure are to be regarded as non-limiting examples and that they can be modified within the scope of the claims.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

While the present disclosure has been described with reference to exemplary embodiments, it will be appreciated that the present disclosure is not limited to what has been described above. For example, it will be appreciated that the dimensions of the parts may be varied as needed. Accordingly, it is intended that the present disclosure may be limited only by the scope of the claims appended hereto.

1-15. (canceled)

16. An actuating system for a medicament delivery device, the actuating system comprising:
a first clamp arranged around a plunger of the medicament delivery device for selectively retaining and releasing the plunger;
a piezoelectric actuator coupled to the first clamp for moving the plunger in a first direction via a plurality of consecutive steps; and
an electric controller configured for controlling the piezoelectric actuator and the first clamp independently to coordinate the moving of the plunger.

17. The actuating system of claim 16, wherein the first clamp is operated by a first piezo stack actuator, and the electric controller is configured to periodically expand and contract the first piezoelectric actuator to retain and release the plunger.

18. The actuating system of claim 16, wherein the piezoelectric actuator includes a second piezo stack actuator, and the electric controller is configured to periodically expand and contract the second piezoelectric stack actuator to move the plunger.

19. The actuating system of claim 16, wherein the electric controller is configured to move the plunger and the first clamp together in the first direction when the plunger is retained by the first clamp.

20. The actuating system of claim 16, wherein the electric controller is configured to move the first clamp without the plunger in a second direction opposite to the first direction when the plunger is released by the first clamp.

21. The actuating system of claim 20, further comprising a plunger holder placed around the plunger and configured to resist the plunger to move in the second direction while the plunger is released by the first clamp, wherein the plunger holder includes a second clamp operated via a third piezo stack actuator controlled by the electric controller.

22. The actuating system of claim 16, wherein the electric controller is operable via at least one of a battery and an AC power source, wherein the electric controller is programmable for moving the plunger at a desired speed;
wherein moving the plunger includes advancing and retracting the plunger.

23. A medicament delivery device, comprising:
a syringe barrel having a reservoir therein for holding a medicament;
a nozzle disposed at a first end of the syringe barrel with at least one opening for allowing the medicament to be expelled from the reservoir;
a plunger received at a second end of the syringe barrel; and
an actuating system for ejecting the medicament from the reservoir, wherein the actuating system comprising:
(i) a first clamp arranged around the plunger for selectively retaining and releasing the plunger;
(ii) a piezoelectric actuator operatively connected to the plunger for moving of the plunger in a first direction via a plurality of consecutive steps; and
(iii) an electric controller configured for controlling the piezoelectric actuator and the first clamp independently to coordinate the moving of the plunger.

24. The medicament delivery device of claim 23, wherein the electric controller is configured to move the plunger and the first clamp together in the first direction when the plunger is retained by the first clamp.

25. The medicament delivery device of claim 23, wherein the electric controller is configured to move the first clamp without the plunger in a second direction opposite to the first direction when the plunger is released by the first clamp.

26. The medicament delivery device of claim 23, wherein the first clamp is operated by a first piezo stack actuator, and the electric controller is configured to periodically expand and contract the first piezoelectric actuator to retain and release the plunger.

27. The medicament delivery device of claim 23, wherein the piezoelectric actuator includes a second piezo stack actuator, and the electric controller is configured to periodically expand and contract the second piezoelectric actuator to move the plunger.

28. The medicament delivery device of claim 23, further comprising a plunger holder placed around the plunger and configured to resist the plunger to move in a second direction opposite to the first direction while the plunger is released by the first clamp, wherein the plunger holder includes a second clamp operated via a third piezo stack actuator controlled via the electric controller.

29. The medicament delivery device of claim 28, wherein the electric controller is configured to control the first and second clamp such that when the first clamp releases the plunger, second clamp retains the plunger, and when the first clamp retains the plunger, second clamp releases the plunger.

30. The medicament delivery device of claim 23, wherein the electric controller is programmable for moving the plunger at a desired speed, wherein moving the plunger includes advancing and retracting the plunger.

31. An actuating system for a medicament delivery device, the actuating system comprising:
a first clamp operatively engaged with a plunger of the medicament delivery device in a retained position and in a. released position;
a first piezoelectric actuator coupled to the first clamp for moving the plunger in a first direction;
a second piezoelectric actuator; and
an electric controller that expands and retracts the second piezoelectric actuator to move the clamp between the retained and released positions and moves the plunger and the first clamp together in the first direction when the first clamp is in the retained position.

32. The actuating system of claim 31, wherein the electric controller moves the first clamp relative to the plunger in a second direction that is opposite to the first direction when the first clamp is in the released position.

33. The actuating system of claim 31, wherein a battery powers the electric controller.

34. The actuating system of claim 31, wherein the electric controller is programmable such that the plunger is moved at a desired speed during advancing and retracting the plunger.
